(19) United States
(12) Patent Application Publication
(10) Pub. No.: US 2026/0293921 A1
(43) Pub. Date: Oct. 1, 2026

(54) METHOD FOR PRODUCING YOGURT

(71) Applicant: MEIJI HOLDINGS CO., LTD., Tokyo (JP)

(72) Inventors: Satoru OZAKI, Tokyo (JP); Yoshiko HONME, Tokyo (JP); Masashi MORIFUJI, Tokyo (JP)

(73) Assignee: MEIJI HOLDINGS CO., LTD., Tokyo (JP)

(21) Appl. No.: 19/475,972

(22) PCT Filed: Apr. 26, 2024
(86) PCT No.: PCT/JP2024/016459

§ 371 Date: Oct. 16, 2025

(30) Foreign Application Priority Data
Apr. 28, 2023 (JP) 2023-074942

(51) Int. Cl. A23C 9/13; A23C 9/123; A61K 9/00; A61K 31/706

(52) U.S. Cl. CPC A23C 9/1322; A23C 9/1234; A23C 9/1238; A61K 9/0056; A61K 31/706

(57) ABSTRACT

A method for producing yogurt, comprising a culturing step of culturing at least one NMN-producing lactic acid bacterium selected from the group consisting of Lactobacillus delbrueckii and Limosilactobacillus reuteri in an NAD-containing medium prepared by adding nicotinamide adenine dinucleotide to a milk component-containing medium, and obtaining a yogurt containing nicotinamide mononucleotide.

### TECHNICAL FIELD

The present invention relates to a method for producing yogurt, and more specifically, relates to a method for producing yogurt containing nicotinamide mononucleotide.

### BACKGROUND ART

Nicotinamide mononucleotide (NMN) is a precursor of nicotinamide adenine dinucleotide (NAD) and is a substance that has been receiving particular attention in recent years.

For example, maintenance of the activity of sirtuins, which are called longevity genes, is considered important for the control of aging, but a decrease in the tissue concentration of NAD required for that activity accompanies getting older (aging); since this is considered a cause of aging, the administration or intake of NMN, which is a precursor of NAD, is said to bring about an anti-aging effect.

More specifically, for example, Mills Kathryn F. et al., Cell metabolism, 24.6, 2016, p. 795-806 (NPL 1) reports that administration of NMN to aged mice confirmed the suppression of obesity due to aging, the improvement of energy metabolism, and the improvement of insulin resistance, lipid metabolism, eye function, bone density, and immune function, and resulted in the suppression of changes in gene expression due to aging in skeletal muscle, fat, and liver. Furthermore, type II diabetes is a lifestyle-related disease caused in part by a decrease in glucose tolerance due to aging, but Jun Yoshino et al., Cell Metab., 2011 Oct. 5, 14 (4), p. 528-536 (NPL 2) reports that in mice induced with type II diabetes by high-fat diet administration, administration of NMN confirmed the improvement of impaired glucose tolerance and hepatic insulin sensitivity, and confirmed the recovery of gene expression related to oxidative stress, inflammatory response, and circadian rhythm.

In addition, for example, Luis Rajman et al., Cell Metab., 2018 Mar. 6, 27(3), p. 529-547 (NPL 3) and Xiaonan Wang et al., Brain Research, Volume 1643, 15 Jul. 2016, p. 1-9 (NPL 4) report that the administration of NMN improved liver function, kidney function, skeletal muscle function, and heart function, which had declined due to aging, as well as Alzheimer's type dementia. Furthermore, for example, Zhu et al., Signal Transduction and Targeted Therapy, 2017, e17017 (NPL 5) reports that the administration of NMN to aged mice improved constipation.

For that reason, supplements and the like for ingesting NMN have been commercially available in recent years. However, a method for easily producing an industrially sufficient amount of NMN has not yet been established, and because it is difficult to obtain raw materials, there was a problem of being expensive for daily intake.

As methods for producing NMN, in addition to production by chemical synthesis, a method of producing it using nicotinamide riboside (NR) as a raw material, method of producing it with Escherichia coli, a method of producing it with yeast, and the like are known. Furthermore, International Publication No. WO 2022/138656 (PTL 1) describes a method for producing NMN that includes a step of reacting a specific lactic acid bacterium belonging to the genus Fructobacillus in a reaction solution with a PH of 4 to 10, and International Publication No. WO 2021/070829 (PTL 2) describes that a similar lactic acid bacterium produces NMN and NR.

### CITATION LIST

### Patent Literature

[PTL 1] International Publication No. WO 2022/138656
[PTL 2] International Publication No. WO 2021/070829

### Non Patent Literature

[NPL 1] Mills Kathryn F. et al., Cell metabolism, 24.6, 2016, p. 795-806
[NPL 2] Jun Yoshino et al., Cell Metab. 2011 Oct. 5, 14(4), p. 528-536
[NPL 3] Luis Rajman et al., Cell Metab., 2018 Mar. 6, 27(3), p. 529-547
[NPL 4] Xiaonan Wang et al., Brain Research, Volume 1643, 15 Jul. 2016, p. 1-9
[NPL 5] Zhu et al., Signal Transduction and Targeted Therapy, 2017, e17017

### SUMMARY OF INVENTION

### Technical Problem

Since lactic acid bacteria are microorganisms having a rich dietary history, if it were possible to produce NMN with lactic acid bacteria, and particularly to produce NMN-containing yogurt as yogurt with a long-standing dietary history, it would be desirable from a safety perspective. However, there was a problem in that it is still difficult to produce a sufficient amount of NMN with lactic acid bacteria by conventional methods.

The present invention was made in view of the problems of the above-described conventional art, and an object thereof is to provide a method for producing yogurt that enables the production of yogurt containing a sufficient amount of nicotinamide mononucleotide (NMN) using Lactobacillus delbrueckii and Limosilactobacillus reuteri.

### Solution to Problem

The present inventors conducted diligent research to achieve the above object, and first, when they searched for bacterial species that produce NMN from among many lactic acid bacteria, they found that NMN is produced, albeit at a relatively low concentration, in Lactobacillus delbrueckii and Limosilactobacillus reuteri. Next, as a result of conducting studies to improve the amount of NMN production by these lactic acid bacteria, they found that by adding NAD to a medium and culturing, the production amount of NMN significantly increases, and furthermore, they found that even among the above lactic acid bacteria, strains that did not produce NMN without the addition of NAD come to produce NMN. In addition, they also found that by adding NAD to a milk component-containing medium and culturing (fermenting), it is possible to produce yogurt containing a sufficient amount of NMN, and have thereby completed the present invention.

Aspects of the present invention obtained from such findings are as follows.
[1]

A method for producing yogurt, comprising a culturing step of culturing at least one NMN-producing lactic acid bacterium selected from the group consisting of Lactobacillus delbrueckii and Limosilactobacillus reuteri in an NAD-containing medium prepared by adding nicotinamide adenine dinucleotide to a milk component-containing medium, and obtaining a yogurt containing nicotinamide mononucleotide.
[2]

The method for producing yogurt according to [1], wherein a content of nicotinamide mononucleotide derived from the NAD-containing medium and the lactic acid bacterium in the yogurt is 5 ng/ml or more.
[3]

The method for producing yogurt according to [1] or [2], wherein a content of milk components in the milk Component-containing medium is 5 to 35 w/we in terms of a content of non-fat milk solids.
[4]

The method for producing yogurt according to any one of [1] to [3], wherein a temperature in the culturing is 25 to 55° C.
[5]

The method for producing yogurt according to any one of [1] to [4], wherein a content of nicotinamide adenine dinucleotide in the NAD-containing medium is 1×10² to 1×10⁸ ng/ml relative to a total amount of the NAD-containing medium.
[6]

The method for producing yogurt according to any one of [1] to [5], wherein the addition of nicotinamide adenine dinucleotide to the medium is the addition of Streptococcus thermophilus, which produces nicotinamide adenine dinucleotide in the medium.
[7]

A method for producing nicotinamide mononucleotide, comprising a culturing step of culturing at least one NMN-producing lactic acid bacterium selected from the group consisting of Lactobacillus delbrueckii and Limosilactobacillus reuteri in an NAD-containing medium prepared by adding nicotinamide adenine dinucleotide to a medium.
[8]

The method for producing nicotinamide mononucleotide according to [7], wherein the medium is a milk component-containing medium.
[9]

The method for producing nicotinamide mononucleotide according to [8], wherein a content of milk components in the milk component-containing medium is 5 to 35 w/w % in terms of a content of non-fat milk solids.
[10]

The method for producing nicotinamide mononucleotide according to any one of [7] to [9], wherein a temperature in the culturing is 25 to 55° C.
[11]

The method for producing nicotinamide mononucleotide according to any one of [7] to [10], wherein a content of nicotinamide adenine dinucleotide in the NAD-containing medium is 1×10² to 1×10⁸ ng/ml relative to a total amount of the NAD-containing medium.
[12]

The method for producing nicotinamide mononucleotide according to any one of [7] to [11], wherein the addition of the nicotinamide adenine dinucleotide to the medium is the addition of at least one Selected from the group consisting of Streptococcus thermophilus, which produces nicotinamide adenine dinucleotide in the medium, and a bacterium belonging to the genus Bifidobacterium.
[13]

A method for improving a nicotinamide mononucleotide production amount, comprising a step of adding nicotinamide adenine dinucleotide to a medium for culturing at least one NMN-producing lactic acid bacterium selected from the group consisting of Lactobacillus delbrueckii and Limosilactobacillus reuteri to make an NAD-containing medium.
[14]

The method for improving a nicotinamide mononucleotide production amount according to [13], wherein the medium is a milk component-containing medium.
[15]

The method for improving a nicotinamide mononucleotide production amount according to [14], wherein a content of milk components in the milk component-containing medium is 5 to 35 w/w % in terms of a content of non-fat milk solids.
[16]

The method for improving a nicotinamide mononucleotide production amount according to any one of [13] to [15], wherein the culturing is performed at a temperature of 25 to 55° C.
[17]

The method for improving a nicotinamide mononucleotide production amount according to any one of [13] to [16], wherein an addition amount of the nicotinamide adenine dinucleotide is an amount such that a content thereof is 1×10² to 1×10⁸ ng/ml relative to a total amount of the NAD-containing medium.
[18]

The method for improving a nicotinamide mononucleotide production amount according to any one of [13] to [17], wherein the addition of the nicotinamide adenine dinucleotide to the medium is the addition of at least one selected from the group consisting of Streptococcus thermophilus, which produces nicotinamide adenine dinucleotide in the medium, and a bacterium belonging to the genus Bifidobacterium.

### Advantageous Effects of Invention

According to the present invention, it becomes possible to provide a method for producing yogurt that enables the production of yogurt containing a sufficient amount of nicotinamide mononucleotide (NMN) using Lactobacillus delbrueckii and Limosilactobacillus reuteri. Furthermore, according to the present invention, it also becomes possible to provide an NMN production method capable of producing a sufficient amount of NMN, and an NMN production amount improvement method capable of improving the amount of NMN production by the lactic acid bacteria.

### BRIEF DESCRIPTION OF DRAWINGS

FIG. 1 is a graph showing the NMN concentration (ng/mL) in SMY medium after culturing Lactobacillus delbrueckii, Limosilactobacillus reuteri, and Streptococcus thermophilus shown in Table 1 in the SMY medium.

FIG. 2 is a graph showing the NMN concentration (ng/ML) in SMY+NAD medium after culturing Lactobacillus delbrueckii, Limosilactobacillus reuteri, and Streptococcus thermophilus shown in Table 1 in the SMY+NAD medium.

FIG. 3 is a graph showing the NMN concentration (ng/ml) in SMY medium after culturing the combinations of Limosilactobacillus reuteri and Streptococcus thermophilus shown in Table 3 in the SMY medium.

FIG. 4 is a graph showing the NMN concentration (ng/ml) in SMY medium after culturing the combinations of Lactobacillus delbrueckii and Streptococcus thermophilus shown in Table 4 in the SMY medium.

FIG. 5 is a graph showing the NMN concentration (ng/ml) in SMY medium after culturing a combination of Lr MHD202317 and St MHD202312 at 37° C. or 43° C. in SMY medium of each milk component concentration.

FIG. 6 is a graph showing the NMN concentration (ng/ml) in SMY medium after culturing a combination of Lb MHD202324 and St MHD202312 at 37° C. or 43° C. in SMY medium of each milk component concentration.

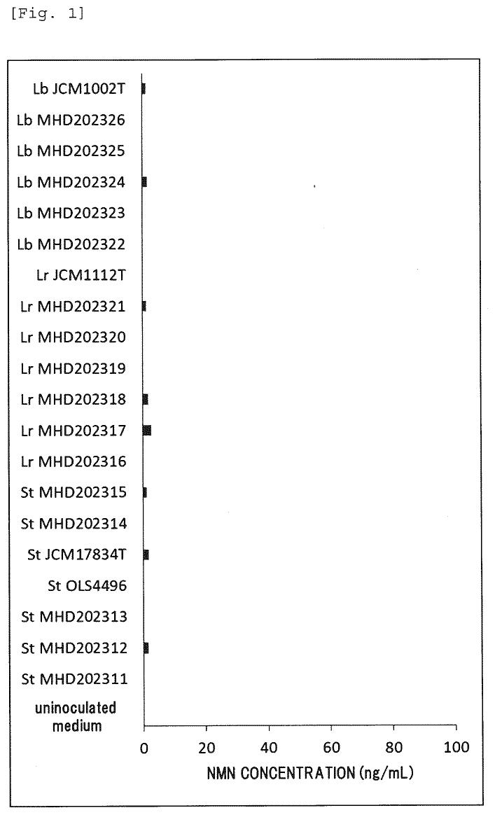

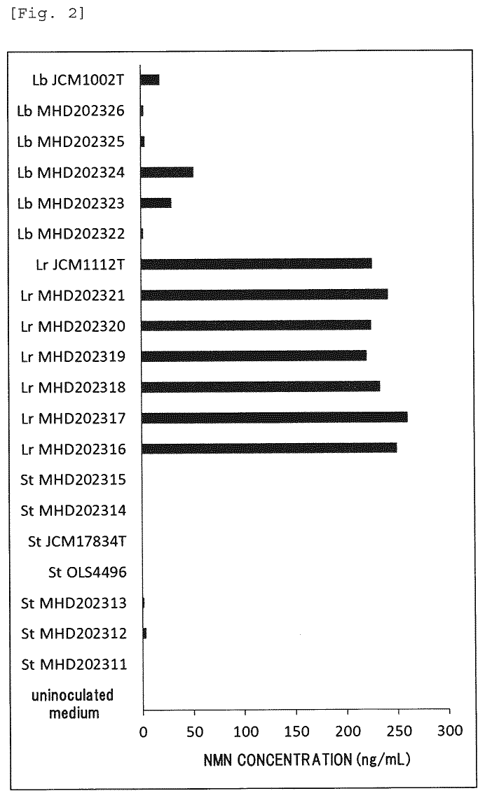

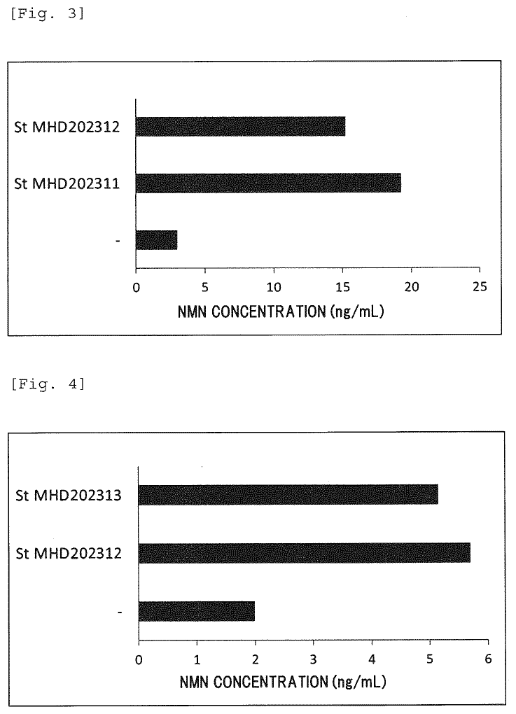

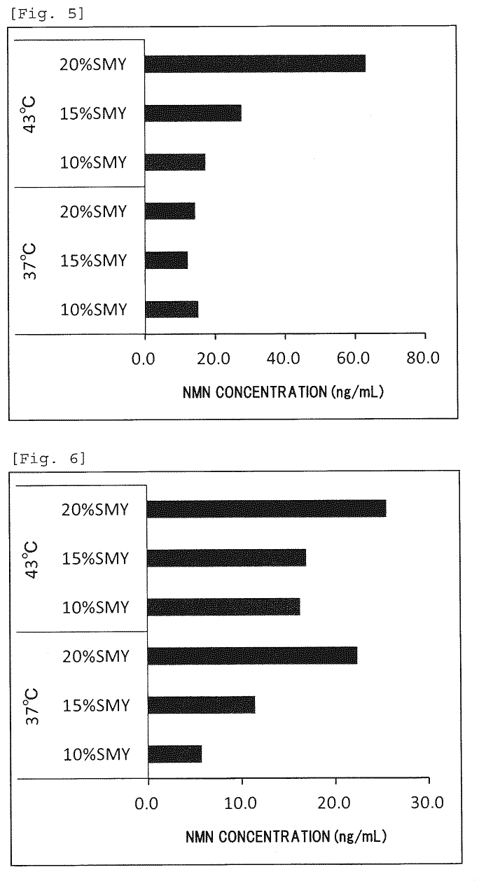

### DESCRIPTION OF EMBODIMENTS

Hereinafter, the present invention will be described in detail in accordance with preferred embodiments thereof.

The present invention provides a method for producing nicotinamide mononucleotide (in this specification, sometimes referred to as “NMN production method”), Comprising a culturing step of culturing at least one NMN-producing lactic acid bacterium selected from the group consisting of Lactobacillus delbrueckii and Limosilactobacillus reuteri in an NAD-containing medium prepared by adding nicotinamide adenine dinucleotide to a medium.

The present invention also provides a method for improving a nicotinamide mononucleotide production amount (in this specification, sometimes referred to as “NMN production amount improvement method”), comprising a step of adding nicotinamide adenine dinucleotide to a medium for culturing at least one NMN-producing lactic acid bacterium selected from the group consisting of Lactobacillus delbrueckii and Limosilactobacillus reuteri to make an NAD-containing medium.

Furthermore, the present invention also provides a method for producing yogurt (in this specification, sometimes referred to as “yogurt production method”), comprising a culturing step of, by using a milk component-containing medium as the medium, culturing at least one NMN-producing lactic acid bacterium selected from the group consisting of Lactobacillus delbrueckii and Limosilactobacillus reuteri in an NAD-containing medium prepared by adding nicotinamide adenine dinucleotide to the milk component-containing medium, and obtaining yogurt containing nicotinamide mononucleotide.

### (NMN)

“Nicotinamide mononucleotide” indicates β-nicotinamide mononucleotide, and is also abbreviated as “NMN.” β-Nicotinamide mononucleotide is an intermediate metabolite when nicotinamide and nicotinamide riboside (NR) are converted to nicotinamide adenine dinucleotide (NAD) in vivo. The present inventors found that NMN is produced by the below-described Lactobacillus delbrueckii and Limosilactobacillus reuteri, and that the addition of nicotinamide adenine dinucleotide enhances the production amount of such NMN or expresses the production ability, and has an excellent NMN production ability-improving action.

### (NAD)

“Nicotinamide adenine dinucleotide” is a substance consisting of NMN and adenosine monophosphate (AMP), and is known to play a central role as a redox coenzyme in dehydrogenation reactions in vivo. In the present invention, nicotinamide adenine dinucleotide may be an oxidized form (NAD⁺), a reduced form (NADH), or a mixture thereof. In this specification, these are sometimes collectively referred to as “NAD.”

### NMN-Producing Lactic Acid Bacterium

In the present invention, the lactic acid bacteria that produce NMN, that is, have NMN-producing ability, are Lactobacillus delbrueckii and Limosilactobacillus reuteri. These may be only one species or a combination of both two species. In this specification, at least one lactic acid bacterium having NMN-producing ability selected from the group consisting of Lactobacillus delbrueckii and Limosilactobacillus reuteri is sometimes collectively referred to as an “NMN-producing lactic acid bacterium” (or “NMN-producing lactic acid bacteria”).

### Lactobacillus delbrueckii

Lactobacillus delbrueckii is a Lactobacillus genus lactic acid bacterium belonging to the family Lactobacillusese. Lactobacillus delbrueckii according to the present invention also includes its subspecies, Lactobacillus delbrueckii ssp.

Examples of the Lactobacillus delbrueckii subspecies include Lactobacillus delbrueckii ssp. bulgaricus, Lactobacillus delbrueckii ssp. delbrueckii, Lactobacillus delbrueckii ssp. lactis, Lactobacillus delbrueckii Ssp. indicus, Lactobacillus delbrueckii ssp. sunkii, and Lactobacillus delbrueckii ssp. jakobsenii.

As for Lactobacillus delbrueckii according to the present invention, it may be only one of them or a combination of two or more, but among them, Lactobacillus delbrueckii ssp. bulgaricus (also called “Bulgarian bacterium”) is particularly preferable.

Lactobacillus delbrueckii according to the present invention is not particularly limited as long as it has NMN-producing ability, that is, in the present invention, it is capable of producing NMN in the below-described NAD-containing medium; for example, when 1×10⁴ to 1×10⁸ cfu/g is inoculated into SMY+NAD medium (10 w/w% skim milk powder, 0.1 w/w yeast extract, 5,000 ng/ML NAD⁺) and anaerobically cultured at 37° C. for 12 hours, one that produces 2 ng/ml or more, more preferably 10 ng/ml or more, still more preferably 30 ng/ml or more, and even more preferably 50 ng/ml or more of NMN in the SMY+NAD medium after culturing can be selected. In the present invention, NMN can be detected and measured, for example, by LC-MS/MS analysis, and as for the conditions, for example, the conditions described in the Examples below can be adopted.

Lactobacillus delbrueckii according to the present invention is not particularly limited, but more specific examples include Lactobacillus delbrueckii ssp. bulgaricus JCM 1002^{T} strain (Lb JCM1002T strain) and the like, and the strain may be a subculture thereof, or an artificial mutant, natural mutant, genetically modified strain, derivative strain, Or the like of the strain or a subculture thereof, within a range that does not impair the effects of the present invention. In this specification, strains indicated by a JCM strain number are strains available from the Japan Collection of Microorganisms (JCM), RIKEN BioResource Research Center (http://jom.brc.riken.jp/ja/). Furthermore, Lactobacillus delbrueckii according to the present invention may be a lactic acid bacterium isolated from commercially available fermented milk with a known agar medium for the genus Lactobacillus (e.g., MRS agar, Plate Count Agar with BCP).

### Limosilactobacillus reuteri

Limosilactobacillus reuteri is a Limosilactobacillus genus lactic acid bacterium belonging to the family Lactobacillaceae. As for Limosilactobacillus reuteri according to the present invention, it may be only one of them or a combination of two or more.

Limosilactobacillus reuteri according to the present invention is not particularly limited as long as it has NMN-producing ability, that is, in the present invention, it is capable of producing NMN in the below-described NAD-containing medium; for example, when 1×10⁴ to 1×10⁸ cfu/g is inoculated into SMY+NAD medium (10 w/w % skim milk powder, 0.1 w/w yeast extract, 5,000 ng/ML NAD⁺) and anaerobically cultured at 37° C. for 12 hours, one that produces 10 ng/ml or more, more preferably 100 ng/ml or more, and still more preferably 200 ng/ml or more of NMN in the SMY+NAD medium after culturing can be selected.

Limosilactobacillus reuteri according to the present invention is not particularly limited, but more specific examples include Limosilactobacillus reuteri JCM 1112T strain (Lr JCM1112T strain) and the like, and the strain may be a subculture thereof, or an artificial mutant, natural mutant, genetically modified strain, derivative strain, or the like of the strain or a subculture thereof, within a range that does not impair the effects of the present invention. Furthermore, Limosilactobacillus reuteri according to the present invention may be a lactic acid bacterium isolated from commercially available fermented milk with a known agar medium for the genus Lactobacillus (e.g., MRS agar, Plate Count Agar with BCP).

### Medium

In the present invention, when simply referred to as “medium,” it indicates one that does not contain NAD, that is, a medium before the addition of NAD. The medium according to the present invention is not particularly limited as long as it is a medium in which the NMN-producing lactic acid bacterium can be cultured, and examples include MRS medium, GYP medium, and a milk component-containing medium. Among these, a milk component-containing medium is particularly preferable in the NMN production method and the NMN production amount improvement method of the present invention, and a milk component-containing medium is necessary in the yogurt production method of the present invention. In the present invention, when simply referred to as a “milk component-containing medium,” it indicates a medium that contains milk components and does not contain NAD.

In the present invention, the milk components indicate milk and milk products that contain at least non-fat milk solids (that is, in which non-fat milk solids are detectable as the remaining component after subtracting the fat content from the total milk solids), and examples include raw milk (e.g., milk from cows, water buffalo, sheep, goats, etc.), whey, and processed products thereof (e.g., skim milk, skim milk powder, concentrated skim milk, whole milk, whole milk powder, concentrated whole milk, sterilized milk, condensed milk, whey powder, buttermilk, butter, cream, cheese, whey protein concentrate (WPC), whey protein isolate (WPI), α-lactalbumin (α-La), β-lactoglobulin (β-Lg)), and the like, and these may be one species or a combination of two or more.

The milk component-containing medium may consist only of the milk components or may contain the milk components and other components. Examples of the other components include water (e.g., ultrapure water), yeast extract, saccharides, sugar alcohols, minerals, vitamins, proteins, peptides, amino acids, organic acids, PH adjusters, starch and modified starch, dietary fiber, fruits/vegetables and processed products thereof, meat, animal and plant crude drug extracts, naturally derived polymers (collagen, hyaluronic acid, chondroitin, etc.), oils and fats, thickeners, emulsifiers, solvents, surfactants, gelling agents, stabilizers, buffers, suspending agents, viscosity agents, excipients, disintegrants, binders, fluidizers, preservatives, coloring agents, flavoring agents, correctives, sweeteners, and the like, and these may be one species or a combination of two or more. In the present invention, the yeast extract is an extract of the contents of yeast, and as the yeast, for example, Saccharomyces cerevisiae is preferable. The yeast extract contains amino acids, peptides, nucleic acids, minerals, etc., and although their composition differs depending on the type of yeast, culture conditions, extraction conditions, etc., any yeast extract can be used without particular limitation.

The content of the milk components in the milk component-containing medium is preferably 5 to 35 w/w %, more preferably 10 to 23 w/w %, still more preferably 15 to 22 w/w %, and even more preferably 16 to 21 w/w %, relative to the total amount of the milk component-containing medium (not including NAD, the same applies hereinafter), in terms of the content of non-fat milk solids. The content of the non-fat milk solids can be measured by the method described in Japan's “Ministerial Ordinance on Milk and Milk Products Concerning Compositional Standards, etc. (hereinafter, sometimes referred to as ‘Milk Ordinance’).”

When the milk component-containing medium contains the yeast extract, its content is preferably 0.001 to 1 w/w %, and more preferably 0.001 to 0.1 w/w %, relative to the total amount of the milk component-containing medium. The pH of such a milk component-containing medium is usually 6 to 7.

When performing the below-described activation culture, the medium used for the second and subsequent activation cultures and the culturing step may contain part or all of the culture solution after the previous culture. It is preferable that the media are each subjected to a sterilization treatment or pasteurization treatment.

### NAD-Containing Medium

The medium used in the culturing step according to the present invention is a medium to which NAD has been added to the above-described medium (preferably a milk component-containing medium; in the yogurt production method, a milk component-containing medium) (in this specification, sometimes referred to as “NAD-containing medium”). In the NMN production amount improvement method of the present invention, NAD is added to the medium to make the NAD-containing medium. In the NAD-containing medium, the content of NAD is preferably 1×10² to 1×10⁸ ng/ML, more preferably 1×10³ to 1×10⁸ ng/ml, and still more preferably 1×10³ to 1×10⁷ ng/ml, in terms of NAD⁺, relative to the total amount of the NAD-containing medium.

In the present invention, the method for adding NAD to the medium may be a method of preparing the NAD-containing medium by adding NAD⁺ or NADH itself to the medium, but as another aspect, for example, it may be a method of adding an NAD-producing bacterium (preferably, the below-described NAD-producing Streptococcus thermophilus) that produces nicotinamide adenine dinucleotide in the medium to the medium and performing culturing, thereby producing NAD in the medium to make the NAD-containing medium. In this case, the NMN-producing lactic acid bacterium and the NAD-producing bacterium may both be inoculated into the medium simultaneously, or the NAD-producing bacterium may be inoculated first. When the NAD-producing bacterium is inoculated first, the content of NAD in the NAD-containing medium can be adjusted in advance to the content listed above by first culturing the NAD-producing bacterium in the medium. On the other hand, when both are inoculated simultaneously, it is preferable that the content of NAD in the NAD-containing medium reaches the content listed above within 12 hours, preferably within 3 hours, from the start of culturing.

### NAD-Producing Bacterium

Examples of the NAD-producing bacterium according to the present invention include Streptococcus thermophilus, which produces nicotinamide adenine dinucleotide in the medium, and a bacterium belonging to the genus Bifidobacterium, and these may be one of these species or both species.

Streptococcus thermophilus

Streptococcus thermophilus is a Streptococcus also called thermophilus bacterium, and is a lactic acid bacterium capable of producing lactic acid from lactose. As for Streptococcus thermophilus according to the present invention, it may be only one species or a combination of two or more.

Streptococcus thermophilus according to the present invention is not particularly limited as long as it has NAD-producing ability, that is, in the present invention, it is capable of producing NAD in the medium (NAD-producing Streptococcus thermophilus); for example, when 1×10⁶ to 1×10⁸ cfu/g is inoculated into SMY medium (10 w/w % skim milk powder, 0.1 w/w yeast extract) and anaerobically cultured at 37° C. for 12 hours, one that produces 1000 ng/ml or more, and more preferably 3000 ng/ml or more of NAD⁺ in the SMY medium after culturing can be selected. In the present invention, the amount of NAD⁺ can be measured, for example, by LC-MS/MS analysis, and as for the conditions, for example, the conditions described in the Examples below can be adopted. Any Streptococcus thermophilus usually satisfies the above conditions.

More specific examples of Streptococcus thermophilus according to the present invention include, but are not limited to, a bacterial strain such as Streptococcus thermophilus specified by Accession Number NITE BP-02875 (hereinafter, sometimes referred to as “St. OLS4496 strain”). The St OLS4496 strain is deposited as follows: (1) Identification: Streptococcus thermophilusOLS 4496 ; (2) Accession Number: NITE BP-02875; (3) Date of Deposit: Feb. 5, 2019; and (4) Depositary Institution: NITE Patent Microorganisms Depositary (NPMD) National Institute of Technology and Evaluation (Room 122, 2-5-8 Kazusakamatari, Kisarazu-shi, Chiba 292-0818, Japan). The St OLS4496 strain may be a subculture of the strain, or an artificial mutant, natural mutant, genetically modified strain, derivative strain, or the like of the strain or a subculture thereof, within a range that does not impair the effects of the present invention.

### Bacterium Belonging to the Genus Bifidobacterium

Other examples of the NAD-producing bacterium include a bacterium belonging to the genus Bifidobacterium. More specific examples of the bacterium belonging to the genus Bifidobacterium include Bifidobacterium breve JCM1192^{T}, Bifidobacterium longum JCM1217^{T}, Bifidobacterium adolescentis JCM1275^{T}, and Bifidobacterium pseudolongum JCM1205^{T}, and these strains may be subcultures thereof, or an artificial mutant, natural mutant, genetically modified strain, derivative strain, or the like of the strain or a subculture thereof, within a range that does not impair the effects of the present invention. It has been confirmed that these bacteria, when an activation culture solution is inoculated at 1 v/v % into GAM bouillon (Nissui Pharmaceutical CO., Ltd., #05422) and anaerobically cultured at 37° C. for 24 hours, respectively produce 1178, 237, 945, and 947 ng/ml of NAD⁺ in the GAM bouillon after culturing. Furthermore, since other multiple strains of Bifidobacterium also produce 276 to 1390 ng/ml of NAD⁺ in a similar culture, it can be said that any bacterial strain belonging to the genus Bifidobacterium usually has NAD-producing ability.

### Culturing Step

In the NMN production method, the NMN production amount improvement method, and the yogurt production method of the present invention, the NMN-producing lactic acid bacterium is cultured in the NAD-containing medium. In the yogurt production method of the present invention, culturing the NMN-producing lactic acid bacterium in the milk component-containing medium (NAD-containing) is synonymous with fermenting the milk component-containing medium with the NMN-producing lactic acid bacterium.

### Activation Culture

It is preferable that the NMN-producing lactic acid bacterium and, if necessary, the NAD-producing bacterium to be subjected to the culturing according to the present invention are each subjected to an activation culture before the below-described culturing, for example, after low-temperature storage (e.g., 10° C. or lower) or frozen storage. As for such an activation culture, for example, it is preferable to perform culturing in the medium (preferably a milk component-containing medium) at 37° C. under anaerobic conditions for 4 to 40 hours. At this time, the amount of bacteria to be subjected to the activation culture is not particularly limited, but it is preferably an amount such that each bacterial amount is 1 platinum loopful/5 mL relative to the medium, and more specifically, it is preferably an amount such that the viable cell count is 1×10² to 2×10⁸ cfu/g, and more preferably 1×10⁴ to 1×10⁶ cfu/g. In the present invention, the measurement of the viable cell count can be measured, for example, by spreading a suitably diluted lactic acid bacterium-containing liquid on a suitable agar medium, culturing, and counting the number of colonies that appear.

The method of the activation culture is not particularly limited, and examples include static culture under conditions known as anaerobic culture conditions for conventional lactic acid bacteria. As for the anaerobic culture conditions, for example, culturing under a nitrogen aeration condition or culturing using a gas concentration adjuster (e.g., AnaeroPack (registered trademark, the same applies hereinafter) manufactured by Mitsubishi Gas Chemical Company, Inc.) can be adopted. The activation culture may be repeated multiple times (preferably twice).

### Culturing

In the culturing step according to the present invention, the NMN-producing lactic acid bacterium (or the NMN-producing lactic acid bacterium after the activation culture) is inoculated into the NAD-containing medium and cultured.

The inoculation amount of the NMN-producing lactic acid bacterium into the NAD-containing medium (the total if there are two or more species of NMN-producing lactic acid bacteria, the same applies hereinafter) is preferably an amount such that the viable cell count is 1×10⁴ to 1×10⁹ cfu/g, more preferably 1×10⁵ to 1×10⁹ cfu/g, and still more preferably 1×10⁶ to 1×10⁸ cfu/g, relative to the NAD-containing medium.

When Streptococcus thermophilus is added as the NAD-producing bacterium as a method for adding NAD to the medium, more specifically, as the culturing step according to the present invention, the NMN-producing lactic acid bacterium (or the NMN-producing lactic acid bacterium after the activation culture) and Streptococcus thermophilus (or Streptococcus thermophilus after the activation culture) are inoculated into the medium and cultured. The inoculation of Streptococcus thermophilus may be simultaneous with the inoculation of the NMN-producing lactic acid bacterium as described above, or it may be earlier.

When Streptococcus thermophilus is added as the method for adding NAD to the medium, the inoculation amount of the NMN-producing lactic acid bacterium into the medium (the total if there are two or more species of NMN-producing lactic acid bacteria, the same applies hereinafter) is the same as above. The inoculation amount of Streptococcus thermophilus is not particularly limited as long as the NAD content in the resulting NAD-containing medium satisfies the above conditions, but for example, it is preferably an amount such that the viable cell count is 1×10⁴ to 1×10⁹ cfu/g, more preferably 1×10⁵ to 1×10⁹ cfu/g, and still more preferably 1×10⁶ to 1×10⁸ cfu/g, relative to the medium.

The culturing conditions in the culturing step can be appropriately selected according to the growth conditions of each added NMN-producing lactic acid bacterium or NAD-producing bacterium, the amount of the NAD-containing medium or medium, and the like, and are not particularly limited, but the culture temperature is preferably 25 to 55° C., more preferably 30 to 46° C., still more preferably 35 to 45° C., and even more preferably 38 to 44° C.

As for other conditions in the culturing step, conditions known as culture conditions for conventional lactic acid bacteria or fermentation conditions for yogurt can be appropriately adopted, and either aerobic or anaerobic conditions may be used, and either static culture or shaking culture may be used. The culture time is usually 2 to 24 hours, more preferably 3 to 12 hours, and still more preferably 3 to 6 hours. Furthermore, it is preferable to culture until the pH of the culture solution (NAD-containing medium) becomes 5.5 or lower, and more preferable to culture until the pH becomes 4.6 or lower.

### NMN-Containing Composition

By the culturing step, NMN is produced in the NAD-containing medium, so that a culture containing NMN can be

1. A method for producing yogurt, comprising a culturing step of culturing at least one NMN-producing lactic acid bacterium Selected from the group consisting of Lactobacillus delbrueckii and Limosilactobacillus reuteri in an NAD-containing medium prepared by adding nicotinamide adenine dinucleotide to a milk component-containing medium, and obtaining a yogurt containing nicotinamide mononucleotide.

2. The method for producing yogurt according to claim 1, wherein a content of nicotinamide mononucleotide derived from the NAD-containing medium and the lactic acid bacterium in the yogurt is 5 ng/ml or more.

3. The method for producing yogurt according to claim 1, wherein a content of milk components in the milk component-containing medium is 5 to 35 w/w % in terms of a content of non-fat milk solids.

4. The method for producing yogurt according to claim 1, wherein a temperature in the culturing is 25 to 55° C.

5. The method for producing yogurt according to claim 1, wherein a content of nicotinamide adenine dinucleotide in the NAD-containing medium is 1×10² to 1×10⁸ ng/ml relative to a total amount of the NAD-containing medium.

6. The method for producing yogurt according to claim 1, wherein the addition of nicotinamide adenine dinucleotide to the medium is the addition of Streptococcus thermophilus, which produces nicotinamide adenine dinucleotide in the medium.
